(19) United States
(12) Patent Application Publication
(10) Pub. No.: US 2026/0033755 A1
(43) Pub. Date: Feb. 5, 2026

(54) DETECTION DEVICE

(71) Applicant: Japan Display Inc., Tokyo (JP)

(72) Inventors: Atsunori OYAMA, Tokyo (JP); Kento HIMOTO, Tokyo (JP)

(21) Appl. No.: 19/357,987

(22) Filed: Oct. 14, 2025

Related U.S. Application Data
(63) Continuation of application No. PCT/JP2024/014073, filed on Apr. 5, 2024.

(30) Foreign Application Priority Data
Apr. 18, 2023 (JP) 2023-067884

(51) Int. Cl. A61B 5/1455; A61B 5/024; A61B 5/1172; G06V 10/141; G06V 40/13; H10F 77/20

(52) U.S. Cl. CPC A61B 5/14552; A61B 5/02433; A61B 5/1172; G06V 10/141; G06V 40/1318; H10F 77/247

(57) ABSTRACT

According to an aspect, a detection device includes: a photodiode; a first light source and a second light source; a light source drive circuit configured to control lighting of the first and second light sources; and a detection circuit configured to output a sensor value corresponding to a photocurrent output from the photodiode. The detection circuit has readout periods and is configured to measure an integrated value of the photocurrent during each readout period. The light source drive circuit has a first mode in which the first and second light sources are alternately lit during the readout periods and a second mode in which one of the first and second light sources is lit during the readout periods. The readout periods include a first readout period in the first mode and a second readout period having a different length of time from the first readout period in the second mode.

### CROSS-REFERENCE TO RELATED APPLICATION

This application claims the benefit of priority from Japanese Patent Application No. 2023-067884 filed on Apr. 18, 2023 and International Patent Application No. PCT/JP2024/014073 filed on Apr. 5, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-032005). Such optical sensors each include a plurality of photodiodes each formed with an organic semiconductor material as an active layer. As described in International Patent Application Publication No. WO 2020/188959, each of the photodiodes is located between a lower electrode and an upper electrode; and, for example, the lower electrode, an electron transport layer, the active layer, a hole transport layer, and the upper electrode are stacked in this order.

For example, one optical sensor may be used for measurements of various types of biometric information, such as an oxygen saturation level in blood (hereinafter referred to as a “blood oxygen saturation level (SpO₂)”) or an image of a vascular pattern of veins or the like. The optical sensor is required to improve detection accuracy in each of the measurements of different objects to be detected or different biological information.

### SUMMARY

According to an aspect, a detection device includes: a photodiode in which a lower electrode, a lower buffer layer, an active layer, an upper buffer layer, and an upper electrode are stacked in the order as listed; a first light source and a second light source that are configured to emit light to the photodiode; a light source drive circuit configured to control lighting of the first light source and the second light source; and a detection circuit that is coupled to the photodiode and is configured to output a sensor value corresponding to a photocurrent output from the photodiode. The detection circuit has a plurality of readout periods and is configured to measure an integrated value of the photocurrent during each of the readout periods. The light source drive circuit has a first mode in which the first light source and the second light source are alternately lit during the readout periods and a second mode in which one of the first light source and the second light source is lit during the readout periods. The readout periods include a first readout period in the first mode and a second readout period having a different length of time from the first readout period in the second mode.

### BRIEF DESCRIPTION OF THE DRAWINGS

FIG. 1 is a plan view schematically illustrating a detection device according to an embodiment;

FIG. 2 is a sectional view along II-II′ of FIG. 1;

FIG. 3 is a block diagram illustrating an exemplary configuration of the detection device according to the embodiment;

FIG. 4 is a circuit diagram illustrating the exemplary configuration of the detection device according to the embodiment;

FIG. 5 is a timing waveform diagram illustrating an exemplary operation in a first mode of the detection device according to the embodiment;

FIG. 6 is a timing waveform diagram illustrating an exemplary operation in a second mode of the detection device according to the embodiment;

FIG. 7 is a graph for explaining response characteristics of a photodiode; and

FIG. 8 is a graph illustrating portions of first and second regions in FIG. 7 in a magnified way.

### DETAILED DESCRIPTION

The following describes a mode (embodiment) for carrying out the present invention in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

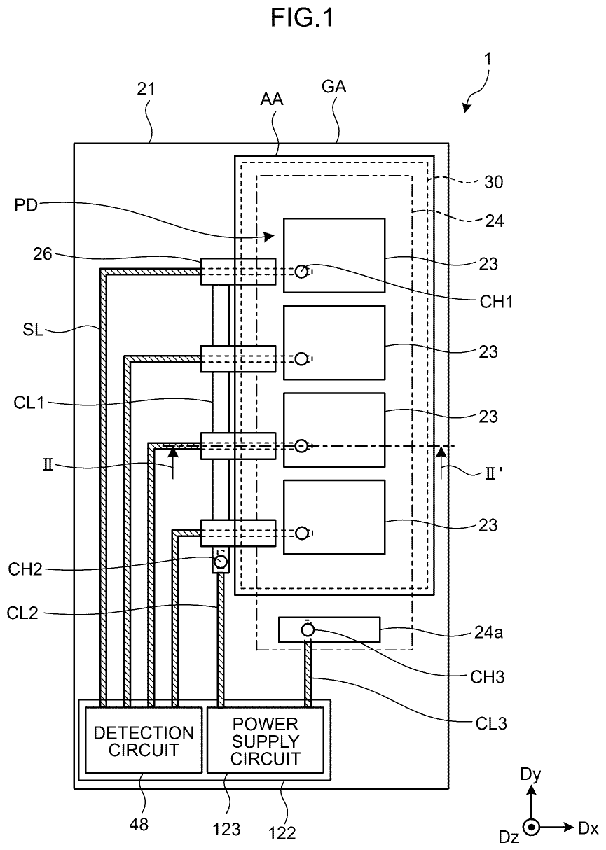

FIG. 1 is a plan view schematically illustrating a detection device according to an embodiment. As illustrated in FIG. 1, a detection device 1 includes a substrate 21, a plurality of photodiodes PD, a plurality of signal lines SL, a plurality of shield layers 26, power supply wiring lines CL1, CL2, and CL3, and a control circuit 122.

The substrate 21 has a detection area AA and a peripheral area GA. The detection area AA is an area provided with the photodiodes PD. The peripheral area GA is an area between the outer perimeter of the detection area AA and the ends of the substrate 21 and is an area not provided with the photodiodes PD. The signal lines SL and the control circuit 122 are provided in the peripheral area GA of the substrate 21.

In the following description, a first direction Dx is one direction in a plane parallel to the substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term “plan view” refers to a positional relation when viewed along a direction orthogonal to the substrate 21.

The detection device 1 includes the photodiodes PD as optical sensor elements. Each of the photodiodes PD outputs an electrical signal in response to light emitted thereto. More specifically, the photodiode PD is an organic photodiode (OPD) including an organic semiconductor. The photodiodes PD are arranged in the second direction Dy in the detection area AA.

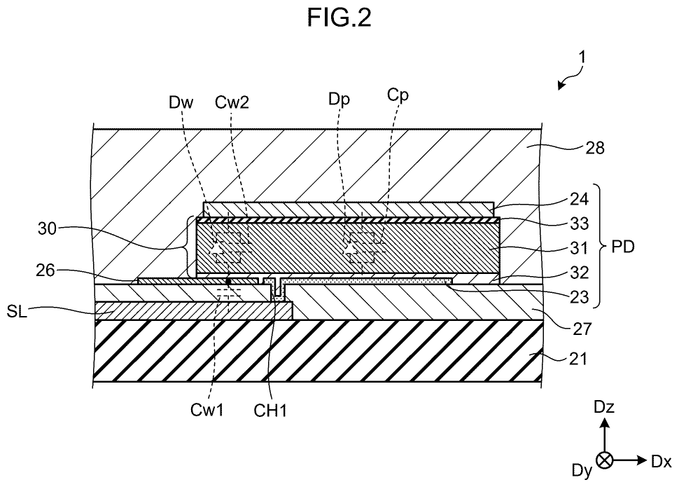

The photodiodes PD each include an organic semiconductor layer 30 (a lower buffer layer 32, an active layer 31, and an upper buffer layer 33 (refer to FIG. 2)), a lower electrode 23 disposed below the organic semiconductor layer 30, and an upper electrode 24 disposed on the upper side of the organic semiconductor layer 30. A plurality of the lower electrodes 23 are provided, one for each of the photodiodes PD, and are arranged in the second direction Dy in the detection area AA. The lower electrodes 23 are arranged apart from one another in the second direction Dy. The organic semiconductor layer 30 and the upper electrode 24 are provided across the photodiodes PD and are provided continuously in the detection area AA. To facilitate viewing of the drawing, FIG. 1 illustrates the organic semiconductor layer 30 and the upper electrode 24 provided on the upper side of the lower electrode 23 with a dashed line and a long dashed double-short dashed line, respectively. The multilayer configuration of the photodiodes PD, the lower electrodes 23, and the upper electrode 24 will be described later with reference to FIG. 2.

The signal lines SL are each electrically coupled to a corresponding one of the lower electrodes 23 of the photodiodes PD. Specifically, in the example illustrated in FIG. 1, the signal lines SL are each coupled to a corresponding one of the lower electrodes 23 through a contact hole CH1 formed in an insulating film 27 (refer to FIG. 2).

Each of the signal lines SL extends in the first direction Dx from a coupling point (contact hole CH1) with the lower electrode 23, bends to the second direction Dy, and extends in the second direction Dy along the arrangement direction of the photodiodes PD. Portions of the signal lines SL extending in the second direction Dy are arranged in the first direction Dx. The signal lines SL are coupled to a detection circuit 48 included in the control circuit 122. In other words, the detection circuit 48 is electrically coupled to the lower electrodes 23 of the photodiodes PD through the signal lines SL.

Each of the signal lines SL and each of the shield layers 26 are provided for a corresponding one of the photodiodes PD. The shield layers 26 are arranged so as to overlap the respective signal lines SL in plan view. In more detail, the shield layers 26 each overlap a portion of a corresponding one of the signal lines SL extending in the first direction Dx and extend in the first direction Dx along the signal lines SL. The shield layers 26 each extend across the detection area AA and peripheral area GA. The shield layers 26 are arranged in the second direction Dy so as to overlap the respective signal lines SL.

The shield layers 26 are coupled to a power supply circuit 123 included in the control circuit 122 via the power supply wiring lines CL1 and CL2 extending in the second direction Dy. More specifically, the power supply wiring line CL1 is provided in the same layer as the shield layers 26 so as to intersect the shield layers 26. As a result, the shield layers 26 are collectively coupled to the same power supply wiring line CL1. The power supply wiring line CL2 is provided in the same layer as the signal lines SL and is electrically coupled to the power supply wiring line CL1 through a contact hole CH2. The power supply wiring line CL2 is electrically coupled to the power supply circuit 123.

With such a configuration, the power supply circuit 123 supplies a reference voltage VCOM to the shield layers 26 via the power supply wiring lines CL1 and CL2. The reference voltage VCOM is a voltage signal having a predetermined fixed potential. The reference voltage VCOM is, for example, a voltage signal having a potential equal to a reference potential Vref supplied to the lower electrodes 23. The reference potential Vref is the predetermined fixed potential. The power supply wiring line CL1 is provided adjacent to the organic semiconductor layer 30 in the first direction Dx. However, the coupling between the shield layers 26 and the power supply circuit 123 may have any configuration, and the arrangement, the number, and the like of the power supply wiring lines CL1 and CL2 can be changed as appropriate.

The upper electrode 24 is provided so as to extend in the second direction Dy across the detection area AA and the peripheral area GA. That is, the upper electrode 24 is provided so as to extend from an area overlapping the organic semiconductor layer 30 to an area not overlapping the organic semiconductor layer 30, and is electrically coupled to the power supply wiring lien CL3 in the area not overlapping the organic semiconductor layer 30. The power supply wiring line CL3 is provided in the same layer as the signal lines SL and is electrically coupled to the upper electrode 24 through a contact hole CH3 and a terminal 24a. The terminal 24a is provided in the same layer as the lower electrode 23.

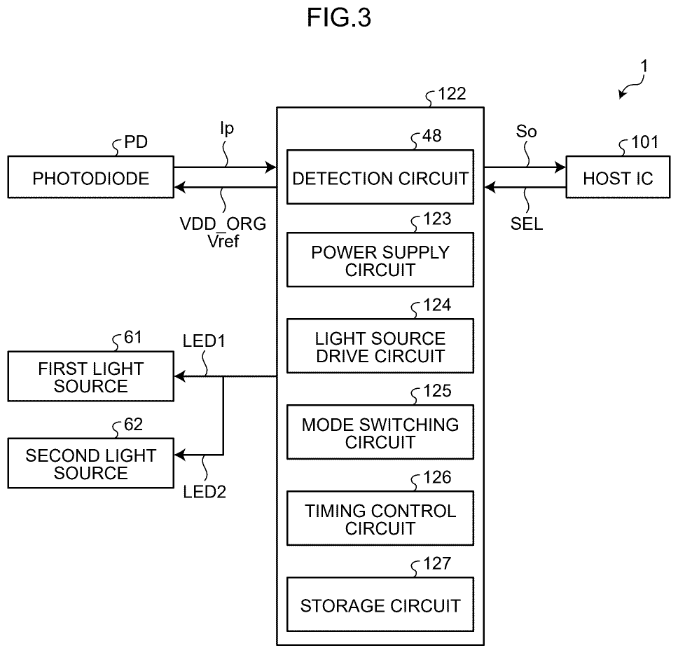

With such a configuration, the upper electrode 24 of the photodiodes PD is coupled to the power supply circuit 123 included in the control circuit 122 via the terminal 24a and the power supply wiring line CL3. The power supply circuit 123 supplies a reference potential VDD_ORG (refer to FIG. 3) to the upper electrode 24 of the photodiodes PD. The reference potential VDD_ORG is a predetermined fixed potential.

The control circuit 122 (detection circuit 48 and power supply circuit 123) is located adjacent to the photodiodes PD in the second direction Dy in the peripheral area GA of the substrate 21. The control circuit 122 is a circuit that controls detection operations by supplying control signals to the photodiodes PD. Each of the photodiodes PD outputs, to the detection circuit 48, the electrical signal in response to the light emitted thereto as a detection signal Vdet. Thereby, the detection device 1 detects information on an object to be detected based on the detection signals Vdet from the photodiodes PD.

A detailed exemplary configuration of the control circuit 122 and the detection operations of the photodiodes PD will be described later with reference to FIGS. 3 to 6. The control circuit 122 (detection circuit 48 and power supply circuit 123) is provided on the same substrate 21 as the photodiodes PD, but is not limited to this configuration. The control circuit 122 (detection circuit 48 and power supply circuit 123) may be provided on another control substrate coupled to the substrate 21, for example, through a flexible printed circuit board or the like. The detection circuit 48 and the power supply circuit 123 may each be formed as an individual circuit.

Although not illustrated in FIG. 1, the detection device 1 includes a first light source 61 and a second light source 62 (refer to FIG. 3). For example, an inorganic light-emitting diode (LED) or an organic electroluminescent (EL) diode (organic light-emitting diode (OLED)) is used as each of the first and the second light sources 61 and 62. The wavelength of light emitted from the first light source 61 is different from that of light emitted from the second light source 62. For example, the first light source 61 emits near-infrared light or infrared light. The second light source 62 emits green light or red light. The green light has a wavelength from 490 nm to 550 nm, for example. The red light has a wavelength from 640 nm to 770 nm, for example. The infrared light has a wavelength from 2500 nm to approximately 25 μm, for example. The near-infrared light has a wavelength from 770 nm to approximately 2500 nm, for example.

The light emitted from the first and the second light sources 61 and 62 is reflected on a surface of the object to be detected, such as a finger, and enters the photodiodes PD. As a result, the detection device 1 can detect a fingerprint by detecting a shape of asperities on the surface of the finger or the like. Alternatively, the light emitted from the first and the second light sources 61 and 62 may be reflected in the finger or the like, or transmitted through the finger or the like, and enter the photodiodes PD. As a result, the detection device 1 can detect information on a living body in the finger or the like. Examples of the information on the living body include, but are not limited to, pulse waves, pulsation, and a vascular image of the finger or a palm. That is, the detection device 1 may be configured as a fingerprint detection device to detect a fingerprint or a vein detection device to detect a vascular pattern of, for example, veins.

The detection device 1 of the present embodiment can detect an oxygen saturation level in blood (hereinafter referred to as a “blood oxygen saturation level (SpO₂)”) in addition to the pulse waves, the pulsation, and the vascular image as the information on the living body based on the light emitted from the first light source 61 and the light emitted from the second light source 62. Thus, the detection device 1 includes the first and the second light sources 61 and 62, and performs the detection based on the light rays having different wavelengths emitted from these light sources, and thereby can detect the various type of information on the living body. The emission colors of the first and the second light sources 61 and 62 described above are examples, and the present disclosure is not limited by the emission colors of the first and the second light sources 61 and 62.

The following describes a multilayer configuration of the photodiode PD and the shield layer 26. FIG. 2 is a sectional view along II-II′ of FIG. 1.

In the following description, a direction from the substrate 21 toward a sealing film 28 in a direction orthogonal to a surface of the substrate 21 is referred to as “upper side” or simply “above”. A direction from the sealing film 28 toward the substrate 21 is referred to as “lower side” or simply “below”.

As illustrated in FIG. 2, the substrate 21 is an insulating substrate and is made using, for example, glass or a resin material. The substrate 21 is not limited to having a flat plate shape and may have a curved surface. In this case, the substrate 21 may be made of a film-like resin.

The signal line SL is provided on the substrate 21. The signal line SL is formed, for example, of metal wiring, and is formed of a material having better conductivity than the lower electrode 23 of the photodiode PD. A portion of the signal line SL (the right end side of the signal line SL in FIG. 2) is provided in a layer between the substrate 21 and the photodiode PD in the third direction Dz. The insulating film 27 is provided on the substrate 21 so as to cover the signal line SL. The insulating film 27 may be an inorganic insulating film or an organic insulating film. The insulating film 27 may be a single layer or a multilayered film.

The photodiode PD is provided on the insulating film 27. In more detail, the photodiode PD includes the lower electrode 23, the lower buffer layer 32, the active layer 31, the upper buffer layer 33, and the upper electrode 24. In the photodiode PD, the lower electrode 23, the lower buffer layer 32, the active layer 31, the upper buffer layer 33, and the upper electrode 24 are stacked in this order in a direction orthogonal to the substrate 21.

The lower electrode 23 is provided on the insulating film 27 and is electrically coupled to the signal line SL through the contact hole CH1 provided in the insulating film 27. The lower electrode 23 is a cathode electrode of the photodiode PD and is formed, for example, of a light-transmitting conductive material such as indium tin oxide (ITO). The detection device 1 of the present embodiment is formed as a bottom-illuminated optical sensor in which the light from the object to be detected passes through the substrate 21 and enters the photodiode PD. The detection device 1 is, however, not limited thereto, and may be a top-illuminated optical sensor.

The active layer 31 changes in characteristics (for example, voltage-current characteristics and resistance value) depending on light emitted thereto. An organic material is used as a material of the active layer 31. Specifically, the active layer 31 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 31, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 31 can be formed by a vapor deposition process (dry process) using any of the low-molecular-weight organic materials listed above. In this case, the active layer 31 may be, for example, a multilayered film of CuPc and F₁₆CuPc, or a multilayered film of rubrene and C₆₀. The active layer 31 can also be formed by a coating process (wet process). In this case, the active layer 31 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 31 can be a film made of a mixture of P3HT and PCBM, or a film made of a mixture of F8BT and PDI.

The lower buffer layer 32 is an electron transport layer and the upper buffer layer 33 is a hole transport layer. The lower buffer layer 32 and the upper buffer layer 33 are provided to facilitate holes and electrons generated in the active layer 31 to reach the lower electrode 23 or the upper electrode 24. The lower buffer layer 32 is in direct contact with the top of the lower electrode 23, and is also provided in areas between the adjacent lower electrodes 23. The active layer 31 is in direct contact with the top of the lower buffer layer 32. The upper buffer layer 33 is in direct contact with the top of the active layer 31, and the upper electrode 24 is in direct contact with the top of the upper buffer layer 33.

Polyethylenimine ethoxylated (PEIE) is used as a material of the electron transport layer. The material of the hole transport layer is a metal oxide layer. For example, tungsten oxide (WO₃) or molybdenum oxide is used as the metal oxide layer.

The materials and the manufacturing methods of the lower buffer layer 32, the active layer 31, and the upper buffer layer 33 are merely exemplary, and other materials and manufacturing methods may be used. For example, each of the lower buffer layer 32 and the upper buffer layer 33 is not limited to a single-layer film, and may be formed as a multilayered film that includes an electron block layer and a hole block layer.

The upper electrode 24 is provided on the upper buffer layer 33. The upper electrode 24 is an anode electrode of the photodiode PD, and is continuously formed over the entire detection area AA. In other words, the upper electrode 24 is continuously provided on the photodiodes PD. The upper electrode 24 faces the lower electrodes 23 with the lower buffer layer 32, the active layer 31, and the upper buffer layer 33 interposed therebetween. The upper electrode 24 is formed, for example, of a light-transmitting conductive material such as ITO or indium zinc oxide (IZO).

The sealing film 28 is provided on the upper electrode 24. An inorganic film, such as a silicon nitride film or an aluminum oxide film, or a resin film, such as an acrylic film, is used as the sealing film 28. The sealing film 28 is not limited to a single layer, and may be a multilayered film having two or more layers obtained by combining the inorganic film with the resin film mentioned above. The sealing film 28 well seals the photodiode PD, and thus can reduce moisture entering the photodiode PD from the upper surface side thereof.

The shield layer 26 is provided in the same layer as the lower electrode 23 on the insulating film 27. The shield layer 26 is formed of the same material as the lower electrode 23, for example, a light-transmitting conductive material such as ITO. However, the shield layer 26 is not limited to this material, and may be formed of a material different from that of the lower electrode 23, for example, a metal material.

The shield layer 26 is disposed with a gap interposed between itself and the lower electrode 23 in the first direction Dx. The shield layer 26 faces the signal line SL with the insulating film 27 interposed therebetween in the third direction Dz. A portion of the shield layer 26 is disposed between the signal line SL and the lower buffer layer 32 of the photodiode PD in the third direction Dz. In other words, the organic semiconductor layer 30 (lower buffer layer 32, active layer 31, and upper buffer layer 33) is provided so as to cover the lower electrode 23 and the portion of the shield layer 26.

The shield layers 26 are supplied with the reference voltage VCOM. As a result, the shield layer 26 reduces parasitic capacitance between the upper electrode 24 of the photodiode PD and the signal line SL, and reduces unintended capacitive coupling between the photodiode PD (upper electrode 24) and the signal line SL.

The detection device 1 of the present embodiment may have a configuration without the shield layer 26. While the example has been described where the lower electrode 23 is a cathode electrode and the upper electrode 24 is an anode electrode, the present disclosure is not limited to this example. The lower electrode 23 may be an anode electrode and the upper electrode 24 may be a cathode electrode. In that case, the lower buffer layer 32 may be a hole transport layer, and the upper buffer layer 33 may be an electron transport layer.

The following describes an exemplary detection method of the detection device 1 of the present embodiment. FIG. 3 is a block diagram illustrating an exemplary configuration of the detection device according to the embodiment. As illustrated in FIG. 3, the control circuit 122 includes the detection circuit 48, the power supply circuit 123, a light source drive circuit 124, a mode switching circuit 125, a timing control circuit 126, and a storage circuit 127.

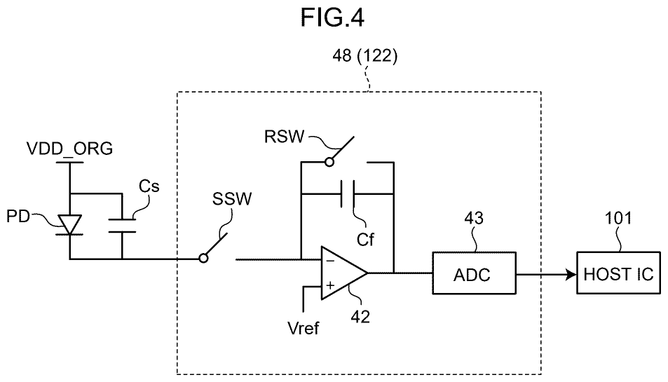

The detection circuit 48 is a current detection circuit that measures current (photocurrent Ip) output from the photodiode PD. The detection circuit 48 is configured, for example, with an operational amplifier circuit 42 and an analog-to-digital (A/D) conversion circuit 43 (refer to FIG. 4). The detection circuit 48 measures the photocurrent Ip output from the photodiode PD, performs signal processing such as an A/D conversion, and outputs a sensor value So corresponding to the photocurrent Ip to a host integrated circuit (IC) 101.

The power supply circuit 123 supplies the reference potential VDD_ORG to the anode of the photodiode PD and also supplies the reference potential Vref to the cathode of the photodiode PD. The reference potential Vref is higher than the reference potential VDD_ORG. As a result, the photodiode PD is driven in a reverse-biased manner.

The light source drive circuit 124 supplies a light source control signal LED1 to the first light source 61 and a light source control signal LED2 to the second light source 62. The light source drive circuit 124 thereby controls lighting and non-lighting of the first and the second light sources 61 and 62. The first and the second light sources 61 and 62 emit light to the photodiode PD based on the light source control signals LED1 and LED2 from the light source drive circuit 124.

The mode switching circuit 125 is a circuit that switches between a detection operation in a first mode M1 and a detection operation in a second mode M2 based on a mode selection signal SEL from the host IC 101. The first mode M1 and the second mode M2 are detection modes set in advance correspondingly to the detection of different biometric information or different objects to be detected. In the present embodiment, the detection device 1 detects the blood oxygen saturation level (SpO₂) in the first mode M1 and detects (images) a vein pattern in the second mode M2.

The detection circuit 48 changes the length of a readout period RD in each of the first mode M1 and the second mode M2 based on a mode switching control signal from the mode switching circuit 125. The light source drive circuit 124 switches the lighting patterns of the first and the second light sources 61 and 62 based on the mode switching control signal from the mode switching circuit 125.

The timing control circuit 126 controls circuits included in the control circuit 122 so as to operate in synchronization or out of synchronization with one another.

The storage circuit 127 temporarily stores therein the sensor value So detected in the first mode M1 and the sensor value So detected in the second mode M2. The storage circuit 127 stores therein in advance various types of information, such as information on the length of the readout period RD for each of the first mode M1 and the second mode M2, and the lighting patterns of the first light source 61 and the second light source 62.

FIG. 4 is a circuit diagram illustrating the exemplary configuration of the detection device according to the embodiment. FIG. 4 schematically illustrates one of the photodiodes PD (refer to FIG. 1).

As illustrated in FIG. 4, the anode of the photodiode PD is supplied with the reference potential VDD_ORG from the power supply circuit 123 (refer to FIG. 1). The cathode of the photodiode PD is coupled to the detection circuit 48. More specifically, the cathode of the photodiode PD is coupled to the inverting input (−) of the operational amplifier circuit 42 via a coupling switch SSW.

Sensor capacitance Cs is coupled in parallel to the photodiode PD. The sensor capacitance Cs is capacitance generated between the upper electrode 24 and the lower electrode 23 of the photodiode PD.

The detection circuit 48 includes the operational amplifier circuit 42, the A/D conversion circuit 43, the coupling switch SSW, and a reset switch RSW. The operational amplifier circuit 42 converts variations in the photocurrent Ip output from the photodiode PD into variations in voltage. The A/D conversion circuit 43 converts analog signals output from the operational amplifier circuit 42 into digital signals. The coupling switch SSW toggles on (coupling) and off (non-coupling) states between the operational amplifier circuit 42 and the photodiode PD. The reset switch RSW is provided to reset an electric charge of a capacitive element Cf of the operational amplifier circuit 42 during a reset period.

When light is emitted to the photodiode PD in an exposure period, a current corresponding to the amount of the light flows through the photodiode PD, which causes an electric charge to be stored in the sensor capacitance Cs. When the coupling switch SSW is turned on in the readout period RD, a current corresponding to the electric charge stored in the sensor capacitance Cs flows to the operational amplifier circuit 42 of the detection circuit 48. The A/D conversion circuit 43 performs signal processing on the voltage signal output from the operational amplifier circuit 42, and outputs the sensor value So corresponding to the photocurrent Ip to the host IC 101. As a result, the detection device 1 can measure the photocurrent Ip output from photodiode PD.

At this point, the reference potential Vref having a fixed potential is applied to the non-inverting input (+) of the operational amplifier circuit 42. When the coupling switch SSW is turned on in the readout period RD, the photodiode PD is coupled to the inverting input (−) of the operational amplifier circuit 42. The cathode of the photodiode PD is at the same reference potential Vref as the non-inverting input (+) due to a virtual short circuit in the operational amplifier circuit 42. The reference potential Vref is higher than the reference potential VDD_ORG. As a result, the photodiode PD is driven in a reverse-biased manner.

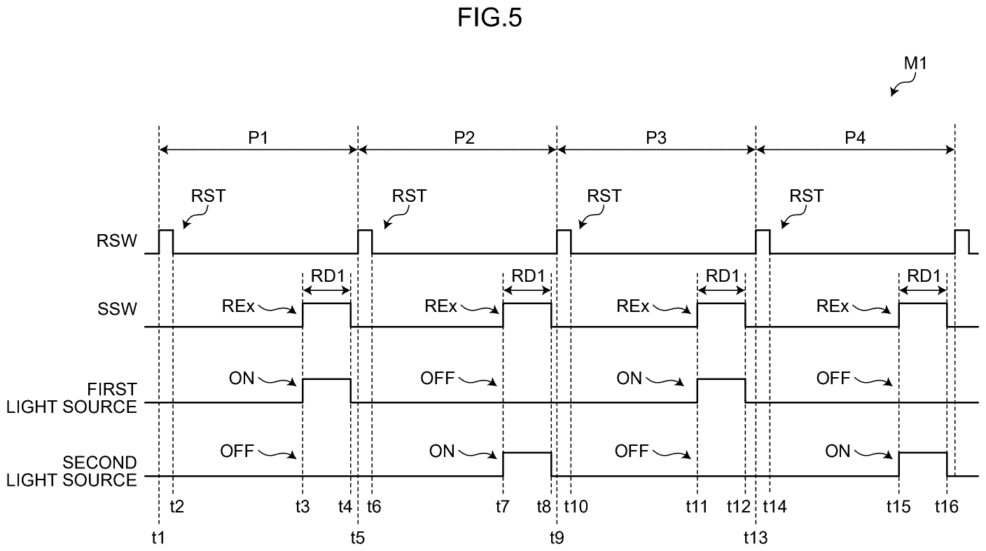

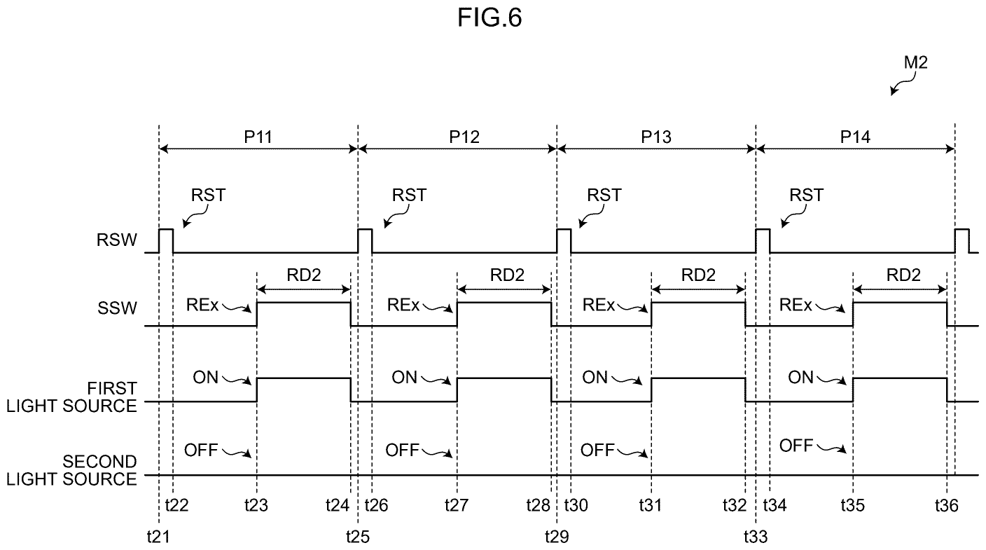

The following describes exemplary operations of the detection device 1 with reference to FIGS. 3 to 6. FIG. 5 is a timing waveform diagram illustrating an exemplary operation in the first mode of the detection device according to the embodiment. FIG. 6 is a timing waveform diagram illustrating an exemplary operation in the second mode of the detection device according to the embodiment.

As illustrated in FIG. 5, the detection device 1 has detection periods P1, P2, P3, and P4 in the first mode M1. The detection device 1 has the reset period during which the reset switch RSW is on and a first readout period RD1 during which the coupling switch SSW is on, in each of the detection periods P1, P2, P3, and P4. In the present embodiment, the exposure period during which the light is emitted to the photodiode PD from the first or the second light source 61 or 62 overlaps the first readout period RD1.

In the following description, the detection periods P1, P2, P3, and P4 may each be simply referred to as a “detection period P” when need not be distinguished from one another.

The light source drive circuit 124 controls lighting and non-lighting of the first and the second light sources 61 and 62 in each of the detection periods P1, P2, P3, and P4. In the first mode M1, the light source drive circuit 124 turns on the first and the second light sources 61 and 62 alternately in the detection periods P1, P2, P3, and P4 (multiple first readout periods RD1).

That is, in the first readout period RD1 of the detection period P1, the first light source 61 is lit and the second light source 62 is unlit. In the first readout period RD1 of the detection period P2, the first light source 61 is unlit and the second light source 62 is lit. In the first readout period RD1 of the detection period P3, the first light source 61 is lit and the second light source 62 is unlit. In the first readout period RD1 of the detection period P4, the first light source 61 is unlit and the second light source 62 is lit.

In the first readout period RD1 of each of the detection periods P1 and P3, the detection circuit 48 measures a photocurrent Ip(NIR) output from the photodiode PD in response to the light emitted from the first light source 61. In the first readout period RD1 of each of the detection periods P2 and P4, the detection circuit 48 measures a photocurrent Ip(R) output from the photodiode PD in response to the light emitted from the second light source 62.

The photocurrent Ip(NIR) is a current component that is output from the photodiode PD in response to light (such as the near-infrared light) emitted from the first light source 61. The photocurrent Ip(R) is a current component of the photocurrent Ip that is output from the photodiode PD in response to light (such as the red light) emitted from the second light source 62.

The detection circuit 48 outputs the sensor value So corresponding to the photocurrent Ip output from the photodiode PD in the first readout period RD1 of each of the detection periods P1, P2, P3, and P4 provided in a time-division manner.

The following describes the detection operations in the detection periods P1 and P2 in detail. The detection period P1 starts at time t1. At time t1, the reset switch RSW is on (coupled state) based on a reset control signal RST from the mode switching circuit 125. As a result, the electric charge of the capacitive element Cf of the operational amplifier circuit 42 is reset. At time t2, the reset switch RSW is off (non-coupled state), and the reset period ends.

At time t3 after a predetermined period of time has elapsed from time t2, the coupling switch SSW is turned on (coupling state) based on a readout control signal REx from the mode switching circuit 125. This operation causes the detection circuit 48 to start the first readout period RD1 of the detection period P1. More specifically, at time t3, the operational amplifier circuit 42 of the detection circuit 48 is coupled to the cathode of the photodiode PD via the coupling switch SSW. At time t3, the first light source 61 is lit and the second light source 62 is unlit based on the light source control signals LED1 and LED2 from the light source drive circuit 124.

In the first readout period RD1 of the detection period P1, the photodiode PD outputs the photocurrent Ip(NIR) in response to light from the first light source 61. The detection circuit 48 measures an integrated value of the photocurrent Ip(NIR) in the first readout period RD1 of the detection period P1. The detection circuit 48 then outputs a sensor value So (NIR) corresponding to the integrated value of the photocurrent Ip(NIR) to the host IC 101.

At time t4 after a predetermined period of time has elapsed from time t3, the coupling switch SSW is turned off (non-coupled state) based on the readout control signal REx from the mode switching circuit 125. This operation causes the detection circuit 48 to end the first readout period RD1 of the detection period P1. Thus, the mode switching circuit 125 controls the length of the first readout period RD1 in the first mode M1 by turning on and off the coupling switch SSW. At time t4, the first and second light sources 61 and 62 are unlit based on the light source control signals LED1 and LED2 from the light source drive circuit 124.

Then, the detection period P2 starts at time t5. At time t5, the reset switch RSW is turned on (coupled state) based on the reset control signal RST from the mode switching circuit 125. As a result, the electric charge of the capacitive element Cf of the operational amplifier circuit 42 is reset. At time t6, the reset switch RSW is turned off (non-coupled state), and the reset period ends.

At time t7 after a predetermined period of time has elapsed from time t6, the coupling switch SSW is turned on (coupling state) based on the readout control signal REx from the mode switching circuit 125. This operation causes the detection circuit 48 to start the first readout period RD1 of the detection period P2. At time t7, the first light source 61 is unlit and the second light source 62 is lit based on the light source control signals LED1 and LED2 from the light source drive circuit 124.

In the first readout period RD1 of the detection period P2, the photodiode PD outputs the photocurrent Ip(R) in response to light from the second light source 62. The detection circuit 48 measures an integrated value of the photocurrent Ip(R) in the first readout period RD1 of the detection period P2. The detection circuit 48 then outputs a sensor value So (R) corresponding to the integrated value of the photocurrent Ip(R) to the host IC 101.

At time t8 after a predetermined period of time has elapsed from time t7, the coupling switch SSW is turned off (non-coupled state) based on the readout control signal REx from the mode switching circuit 125. This operation causes the detection circuit 48 to end the first readout period RD1 of the detection period P2. At time t8, the first and second light sources 61 and 62 are unlit based on the light source control signals LED1 and LED2 from the light source drive circuit 124.

Thereafter, the detection device 1 measures the photocurrent Ip in the first readout period RD1 of each of the detection periods P3 and P4. The detection periods P3 and P4 are the same as the detection periods P1 and P2 described above, and will not be described again.

The host IC 101 can calculate the blood oxygen saturation level (SpO₂) using the sensor value So (NIR) by the first light (near-infrared light) and the sensor value So (R) by the second light (red light) that have been acquired in the first mode M1.

In the first mode M1, the length of each of the detection periods P1, P2, P3, and P4 is 200 μs, for example. The length of the first readout period RD1 of each of the detection periods P1, P2, P3, and P4 is, 50 μs, for example.

As illustrated in FIG. 6, the detection device 1 has detection periods P11, P12, P13, and P14 in the second mode M2. The detection device 1 has the reset period during which the reset switch RSW is on and a second readout period RD2 during which the coupling switch SSW is on, in each of the detection periods P11, P12, P13, and P14. In the present embodiment, the exposure period during which the light is emitted to the photodiode PD from the first or the second light source 61 or 62 overlaps the second readout period RD2.

In the following description, the detection periods P11, P12, P13, and P14 may each be simply referred to as a “detection period P” when need not be distinguished from one another.

In the second mode M2, the operation of the reset switch RSW in the reset period and the operation of the coupling switch SSW in the second readout period RD2 (exposure period) are the same as those in the first mode M1 described above. In the second mode M2, the matters described for the first mode M1 will not be described again, and matters different from those for the first mode M1 will be described.

In the second mode M2, the light source drive circuit 124 turns on one of the first and the second light sources 61 and 62 during the second readout periods RD2 of the detection periods P11, P12, P13, and P14. In FIG. 6, the light source drive circuit 124 causes the first light source 61 to be on and leaves the second light source 62 to be off during the second readout periods RD2 of the detection periods P11, P12, P13, and P14. The mode switching circuit 125 controls the length of the second readout period RD2 in the second mode M2 by turning on or off the coupling switch SSW.

In each of the second readout periods RD2 of the detection periods P11, P12, P13, and P14, the photodiode PD outputs the photocurrent Ip(NIR) in response to the light from the first light source 61. The detection circuit 48 measures the integrated value of the photocurrent Ip(NIR) in each of the second readout periods RD2 of the detection periods P11, P12, P13, and P14. The detection circuit 48 then outputs the sensor value So (NIR) corresponding to the integrated value of the photocurrent Ip(NIR) to the host IC 101.

The host IC 101 can image the vascular pattern of veins using the sensor value So (NIR) generated by the first light (near-infrared light) acquired in the second mode M2.

In the second mode M2, the length of each of the detection periods P11, P12, P13, and P14 is 2000 μs, for example. The length of each of the second readout periods RD2 is 1000 μs, for example.

As described above, the light source drive circuit 124 turns on the first and the second light sources 61 and 62 alternately in each of the first readout periods RD1 in the first mode M1. In the second mode M2, the light source drive circuit 124 causes one of the first and the second light sources 61 and 62 (first light source 61 in FIG. 6) to be on during the second readout periods RD2.

The readout periods RD of the detection circuit 48 include the first readout period RD1 in the first mode M1 and the second readout period RD2 in the second mode M2. The first readout period RD1 and the second readout period RD2 have different length of time. In the present embodiment, the second readout period RD2 is longer than the first readout period RD1.

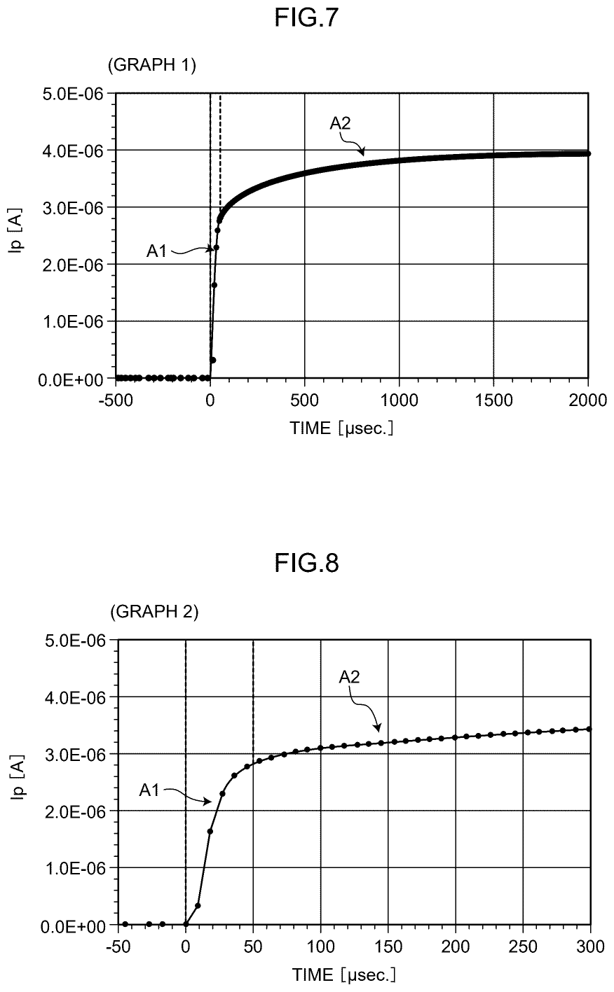

FIG. 7 is a graph for explaining response characteristics of the photodiode. FIG. 8 is a graph illustrating portions of first and second regions in FIG. 7 in a magnified way. In FIGS. 7 and 8, the vertical axis represents the photocurrent Ip output from the photodiode PD. The horizontal axis represents the irradiation time of the light from the light source (first light source 61 or second light source 62), and “0” is the start time at which the first or the second light source 61 or 62 starts lighting.

The response characteristics of the photodiode PD have a first region A1 with a larger gradient of the photocurrent Ip and a second region A2 with a smaller gradient of the photocurrent Ip. In the first region A1, the magnitude (sensitivity) of the photocurrent Ip is smaller than in the second region A2, but the time required for measurement is shorter. In contrast, in the second region A2, the irradiation time of the light from the light source (first or second light source 61 or 62) is longer and the magnitude (sensitivity) of the photocurrent Ip is larger than in the first region A1.

The first readout period RD1 in the first mode M1 described above corresponds to the first region A1 in the response characteristics of the photodiode PD. The second readout period RD2 in the second mode M2 corresponds to the second region A2 in the response characteristics of the photodiode PD.

That is, in the measurement of a blood flow, the blood oxygen saturation level (SpO₂), and the like, the detection device 1 can perform the detection in the first mode M1 using the first region A1 in the response characteristics of the photodiode PD to shorten the measurement cycle. In contrast, in the imaging of the vascular pattern of veins and the measurement of the pulse waves, water content, and so forth, the detection device 1 can perform the detection in the second mode M2 using the second region A2 in the response characteristics of the photodiode PD to increase the sensitivity.

Thus, the detection device 1 can use the response characteristics of the photodiode PD to perform appropriate driving depending on the type of the object to be detected or the type of the biometric information. As a result, the detection device 1 can improve the detection accuracy depending on the type of the object to be detected or the type of the biometric information.

The timing waveform diagrams illustrated in FIGS. 5 and 6 are merely exemplary, and can be changed as appropriate. For example, the first light source 61 emits the near-infrared light and the second light source 62 emits the red light, but is not limited to this example. The first light source 61 may emit infrared light. Alternatively, the second light source 62 may emit green light.

The first and the second light sources 61 and 62 are lit in synchronization with the first readout period RD1 in the first mode M1. The first light sources 61 is lit in synchronization with the second readout period RD2 in the second mode M2. The embodiment is, however, not limited thereto, and the first and the second light sources 61 and 62 only need to be lit at least in the first readout period RD1 in the first mode M1. The period during which the first and the second light sources 61 and 62 are lit may be longer than the first readout period RD1. In other words, the first readout period RD1 may start after a predetermined period of time has elapsed after the first or the second light source 61 or 62 has been lit in the first mode M1. In the second mode M2, the first light source 61 only needs to be lit at least during the second readout period RD2. The period during which the first light source 61 is lit may be longer than the second readout period RD2. In other words, the second readout period RD2 may start after a predetermined period of time has elapsed after the first light source 61 has been lit.

In the second mode M2, the first light source 61 is lit and the second light source 62 is unlit during the second readout periods RD2, but the embodiment is not limited thereto. The first light source 61 may be unlit and the second light source 62 may be lit during the second readout periods RD2.

While the preferred embodiment has been described above, the present invention is not limited to the embodiment described above. The content disclosed in the embodiment is merely an example, and can be variously modified within the scope not departing from the gist of the present disclosure. Any modifications appropriately made within the scope not departing from the gist of the present invention also naturally belong to the technical scope of the present disclosure. At least one of various omissions, substitutions, and changes of the components can be made without departing from the gist of the embodiment and the modifications thereof described above.

What is claimed is:

1. A detection device comprising:
a photodiode in which a lower electrode, a lower buffer layer, an active layer, an upper buffer layer, and an upper electrode are stacked in the order as listed;
a first light source and a second light source that are configured to emit light to the photodiode;
a light source drive circuit configured to control lighting of the first light source and the second light source; and
a detection circuit that is coupled to the photodiode and is configured to output a sensor value corresponding to a photocurrent output from the photodiode, wherein
the detection circuit has a plurality of readout periods and is configured to measure an integrated value of the photocurrent during each of the readout periods,
the light source drive circuit has a first mode in which the first light source and the second light source are alternately lit during the readout periods and a second mode in which one of the first light source and the second light source is lit during the readout periods, and
the readout periods include a first readout period in the first mode and a second readout period having a different length of time from the first readout period in the second mode.

2. The detection device according to claim 1, wherein
the first light source is configured to emit near-infrared light or infrared light, and
the second light source is configured to emit green light or red light.

3. The detection device according to claim 1, wherein the second readout period is longer than the first readout period.

4. The detection device according to claim 1, wherein
the detection circuit comprises an amplifier circuit and a coupling switch that is configured to couple the amplifier circuit to the photodiode, and
a length of the first readout period and a length of the second readout period are controlled by turning on or off the coupling switch.
